(19) United States
(12) Patent Application Publication
(10) Pub. No.: US 2008/0038838 A1
(43) Pub. Date: Feb. 14, 2008

(54) Dioxin-Binding Material and Method of Detecting or Quantifying Dioxin

(75) Inventors: Chikashi Nakamura, Amagasaki-shi (JP); Jun Miyake, Amagasaki-shi (JP); Ikuo Obataya, Amagasaki-shi (JP); Noriyuki Nakamura, Amagasaki-shi (JP); Katsuhisa Shirai, Ichihara-shi (JP); Yasuhiro Inuyama, Hiroshima-shi (JP)

Correspondence Address: FITCH, EVEN, TABIN & FLANNERY, P. O. BOX 18415, WASHINGTON, DC, 20036, US

(73) Assignees: Towa Kagaku Co., Ltd., Hiroshima-shi, Hiroshima (JP); National Institute of Advanced Industrial Science and Technology, Chiyoda-ku, Tokyo (JP)

(21) Appl. No.: 10/574,910

(22) PCT Filed: Oct. 1, 2004
(86) PCT No.: PCT/IB04/03204

§ 371 Date: Feb. 14, 2007

(30) Foreign Application Priority Data
Oct. 10, 2003 (JP) 2003-353026

(51) Int. Cl. C07K 7/06; C07D 319/00; G01N 33/543; G01N 33/566; C07K 7/00

(52) U.S. Cl. 436/501; 436/518; 530/300; 530/329; 530/330; 549/359

(57) ABSTRACT

A principal object of the present invention is to provide low-cost techniques for readily detecting or quantifying dioxins using substances which are inexpensive and can be easily produced, and extracting dioxins using such substances. Dioxin binding peptides of the present invention are highly selective to dioxins, and are therefore capable of detecting or quantifying dioxins in test samples containing impurities. Dioxin binding peptides of the present invention are also capable of selectively extracting dioxins from impurity-containing test samples, and are therefore useful in simple pretreatment for quantifying and analyzing dioxins.

### TECHNICAL FIELD

The present invention relates to an oligopeptide, a linearly-linked peptide and an oligopeptide complex which have affinity to dioxins, as well as to a support linked to any of the oligopeptide, linearly-linked peptide and oligopeptide complex. The present invention also relates to a method of detecting or quantifying dioxins and a method of extracting dioxins, using the oligopeptide, linearly-linked peptide and oligopeptide complex.

### BACKGROUND ART

In recent years, the problem of environmental pollution caused by a variety of chemical substances has worsened, raising concerns about the adverse effects they may have on living organisms. Dioxins, in particular, have such toxicities as carcinogenicity, immunotoxicity, reproductive toxicity and teratogenicity, and therefore, it is required that pollution by dioxins be measured and assessed accurately.

Conventionally, the method of analyzing dioxins with a gas chromatography mass spectrometer has been used as an official method. This method, however, involves great effort due to its troublesome pretreatments and the like, and the analysis also takes a long time, making it difficult to obtain prompt results. Hence, there is an urgent need to develop a highly sensitive analytical method which can be performed quickly and simply at low cost.

The development of techniques utilizing biofunctions as advantageous techniques which can meet the aforementioned requirements is ongoing. Many methods utilizing antibodies as the recognition elements for target substances have been developed as representative methods of detection using biological techniques (e.g., Masako Maeda, “Immunoassay -Rosalyn Yalow's Achievement-”, BUNSEKI, 1999, 839-843) and also practiced (e.g., Masashi Ushiyama, “Analysis of Environmental Samples by Immunoassay”, BUNSEKI, 1998, 736-747; and Masanobu Nakata and Hidero Ohkawa, “Immunoassay for Agricultural Chemicals Using Monoclonal Antibodies”, BUNSEKI, 1999, 492-500).

However, problems with antibodies are their long manufacturing time, expensive cost, and difficulty in obtaining antibodies for low-molecular chemical substances or highly toxic substances.

Moreover, environmental samples such as soil and incinerated ash contain many impurities which adversely affect the detection or chemical analysis of dioxins. For this reason, pretreatment such as soxhlet extraction is usually performed, which requires a great deal of time and effort. If selective binding and extraction of just dioxins from samples containing impurities can be achieved, it will become useful as a simple pretreating method for quantifying and analyzing dioxins. Such a method of extracting dioxins would also be useful in terms of purifying dioxin-contaminated soils, plant wastewater, rivers, etc.

A principal object of the present invention is to provide techniques for readily detecting or quantifying dioxins using substances which are inexpensive and can be easily produced, and for extracting dioxins using such substances.

### DISCLOSURE OF THE INVENTION

The present invention provides an oligopeptide, a linearly-linked peptide and an oligopeptide complex, and a support linked with any of the above for recognizing dioxins, as itemized below. The present invention also provides a method of detecting or quantifying particular dioxin(s) and a method of extracting particular dioxin(s), using any of the aforementioned oligopeptides.

### Item 1:

An oligopeptide represented by Formula (I) shown below:

A₁-Leu-Asp-Gln-A₂-(X)ₙ (I)

where A₁ represents a hydrophobic amino acid residue having a side chain with a cyclic group; A₂ represents a hydrophobic amino acid residue having an aliphatic hydrocarbon group or an aromatic hydrocarbon group; n is zero or one; and X represents an amino acid residue.

### Item 2:

A linearly-linked peptide formed by linking two or more oligopeptides represented by Formula (I) as a repeating unit via a spacer, if necessary.

### Item 3:

An oligopeptide complex formed by using a linker to the C-terminal of the oligopeptide according to Item 1.

### Item 4:

An oligopeptide according to Item 1, wherein A₁ is represented by Formula (II) shown below:

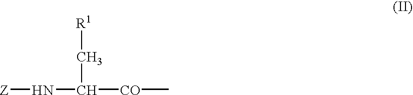

where R¹ represents a cyclic group; and Z represents a hydrogen atom, an alkyl group or an acyl group.

### Item 5:

An oligopeptide according to Item 1, wherein A₁ is phenylalanine, 1-naphthylalanine, or cyclohexylalanine.

### Item 6:

An oligopeptide according to Item 1, wherein A₂ is represented by Formula (III) shown below:

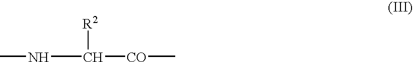

where R² is an alkyl or aryl group.

### Item 7:

An oligopeptide according to Item 1, wherein A₂ is valine, norvaline, leucine, or phenylglycine.

### Item 8:

An oligopeptide according to Item 1, comprising Phe-Leu-Asp-Gln-Ile.

### Item 9:

An oligopeptide according to Item 1, comprising Phe-Leu-Asp-Gln-Val.

### Item 10:

An oligopeptide according to Item 1, comprising Phe-Leu-Asp-Gln-Phg, where Phg represents a phenylglycine residue.

### Item 11:

Use of the oligopeptide, linearly-linked peptide and oligopeptide complex according to any one of Items 1 to 10 for detecting or quantifying dioxin.

### Item 12:

A peptide immobilizing support formed by linking the oligopeptide, linearly-linked peptide and oligopeptide complex according to any one of Items 1 to 10 to a support.

### Item 13:

A peptide immobilizing support according to Item 12, wherein the support is a bead.

### Item 14:

A method of detecting or quantifying dioxin comprising the steps of:
(1) bringing the peptide immobilizing support according to Item 12 into contact with a labeled dummy and a test sample which may contain dioxin; and
(2) detecting or quantifying dioxin based on the amount of the labeled dummy bound to the support which is determined in Step (1).

### Item 15:

A method according to Item 14, wherein the labeled dummy is NBD-labeled 3,4-dichlorophenol.

### Item 16:

A method of extracting dioxin comprising the steps of:
(1) bringing the peptide immobilizing support according to Item 12 into contact with a test sample containing dioxin to bind the dioxin to the support; and
(2) separating the dioxin bound to the support obtained in Step (1) using a solvent.

Throughout the specification, the term “dioxins” denotes polychlorinated dibenzoparadioxins (PCDDs), polychlorinated dibenzofurans (PCDFs), and coplanar PCBs. This is also the same when “dioxins” are simply referred to as “dioxin”.

Moreover, the oligopeptide as represented by General Formula (I), the linearly-linked peptide according to Item 2, and the oligopeptide complex according to Item 3 may collectively be abbreviated as a “dioxin binding peptide” in the specification.

The present inventors prepared a peptide library by a combinatorial chemistry technique, and screened the library, thereby finding peptide sequences capable of binding to dioxins.

### Dioxin Binding Peptide

The present inventors prepared the aforementioned peptide library by split-and-pool synthesis, which is a typical technique employed in combinatorial chemistry (“Combinatorial Chemistry From Basics to Application”; edited by the Japan Combinatorial Chemistry Focus Group; published by Kagaku-dojin, 4/97″), and screened the peptide library by binding them to beads for solid phase peptide synthesis (W. C. Chan and P. D. White, in W. C. Chan P. D. White (Ed.), Fmoc Solid Phase Peptide Synthesis: A Practical Approach, Oxford University Press, New York, 2000, p. 41).

Dioxin binding oligopeptides according to the invention may be prepared by conventional processes such as solid phase synthesis, liquid phase synthesis, etc. The oligopeptides may also be linked to a support (e.g., a bead). In this case, solid phase synthesis is preferably performed on the support beforehand in order to save the trouble of immobilization.

Preferred embodiments of the oligopeptides according to the invention encompass DB1 comprising phenylalanine, leucine, aspartic acid, glutamine, and isoleucine (Phe-Leu-Asp-Gln-Ile; SEQ ID No. 2) from the N-terminal side; and DB2 comprising phenylalanine, leucine, aspartic acid, glutamine, and valine (Phe-Leu-Asp-Gln-Val; SEQ ID No. 3) from the N-terminal side. An oligopeptide comprising phenylalanine, leucine, aspartic acid, glutamine, and phenylglycine (abbreviated as Phg in the specification) from the N-terminal side (Phe-Leu-Asp-Gln-Phg; SEQ ID No. 22) has sensitivity to 2,3,7,8-TeCDD (2,3,7,8-tetrachlorodibenzo-p-dioxin) in a 30% dioxane solvent, which is about ten times higher than that of DB2. This oligopeptide is therefore useful in detecting, quantifying or extracting dioxin.

The amino acid represented by A₁ in Formula (I) may be represented by Formula (II) shown below:

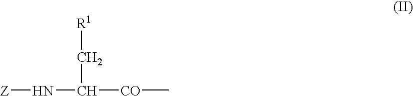

where the cyclic group represented by R¹ may be an aromatic or alicyclic hydrocarbon. Examples of aromatic hydrocarbons include phenyl, toluyl, xylenyl, naphthyl and the like; and examples of alicyclic hydrocarbons include C₃₋₈ alicyclic hydrocaron groups such as cyclopropyl, cyclobutyl, cyclopentyl, cyclohexyl, cycloheptyl, cyclooctyl and the like, among which cyclopentyl and cyclohexyl are preferable.

An aromatic or alicyclic hyrdocarbon may or may not have substituent(s) introduced thereto. Examples of substituents include alkyl groups such as methyl and ethyl groups, methoxy groups, amino groups, methoxycarbonyl groups, nitrile groups (CN), and halogens such as fluorine, chlorine, bromine, and iodine. An aromatic or alicyclic hydrocarbon may have one to three of such substituents, preferably one substituent.

Z in Formula (II) is a hydrogen atom; an alkyl group such as a methyl or ethyl group; or an acyl group such as an acetyl group, and is preferably a hydrogen atom (H). Preferable examples of A₁ include phenylalanine, 1-naphthylalanine, cyclohexylalanine and the like.

Moreover, a natural amino acid, an unnatural amino acid or a group in which two or more amino acids are linked (e.g., peptide) may be added as an acyl group represented by Z in Formula (II). Examples of natural amino acids include asparagine, serine, aspartic acid, glutamine, glutamic acid, threonine, arginine, histidine, glycine, lysine, tyrosine, tryptophan, cysteine, methionine, proline, phenylalanine, alanine, valine, leucine, isoleucine, and the like; and examples of unnatural amino acids include β-alanine, γ-aminobutyric acid, δ-aminopentanoic acid, ε-aminohexanoic acid and the like.

The amino acid represented by A₂ in Formula (I) may be represented by Formula (III) shown below:

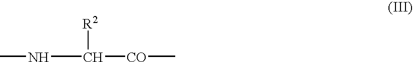

where R² is preferably an aliphatic hydrocarbon group (e.g., sec-butyl, isopropyl, propyl or isobutyl group) or an aromatic hydrocarbon group (e.g., phenyl or naphthyl group). An aromatic or aliphatic hydrocarbon may or may not have substituent(s) introduced thereto. Examples of substituents include alkyl groups such as methyl and ethyl groups, methoxy groups, amino groups, methoxycarbonyl groups, nitrile groups (CN), and halogens such as fluorine, chlorine, bromine, and iodine. An aromatic or aliphatic hydrocarbon may have one to three of such substituents, preferably one substituent.

In one preferred embodiment of the invention, examples of A₂ include valine, norvaline, leucine, phenylglycine and the like.

Examples of X in Formula (I) are natural amino acids such as asparagine, serine, aspartic acid, glutamine, glutamic acid, threonine, arginine, histidine, glycine, lysine, tyrosine, tryptophan, cysteine, methionine, proline, phenylalanine, alanine, valine, leucine, isoleucine and the like; and unnatural amino acids in which the amino group involved in an amide bond is not linked to the alpha carbon, such as β-alanine, γ-aminobutyric acid, δ-aminopentanoic acid, ε-aminohexanoic acid and the like. Preferably, X is an unnatural amino acid. In Formula (I), n is zero or one, and preferably zero.

Most preferably, the oligopeptide according to the invention comprises, from the N-terminal, leucine as a second residue, aspartic acid as a third residue, and glutamine as a fourth residue. Moreover, such an oligopeptide preferably comprises L-amino acids only, because they exhibit a high binding capability with dioxin.

The oligopeptide for use in the invention may be linked to a support in a variety of forms. Useful supports include beads, fibers, sheets and the like, and each of such supports may take a variety of forms. For example, in one preferred embodiment of the invention, a hydrophobic polymer such as polystyrene or the like having a diameter of 1 to 350 μm, preferably 10 to 150 μm, and a substitution rate of about 0.1 to about 1.0 mmol/g, preferably about 0.2 to about 0.3 mmol/g, is preferably used. Such a support is particularly preferable in bead form.

Embodiments of immobilizing the dioxin binding peptide of the present invention to a support encompass immobilizing the N- or C-terminal of the dioxin binding peptide shown in General Formula (I) to the support either directly or via a spacer. Examples of the spacer may include a polyethylene oxide chain. The support immobilizing the dioxin binding peptide of the invention is preferably such that the oligopeptide is attached to the support at the C-terminal with a spacer interposed therebetween.

In another embodiment of the invention, the oligopeptide shown in Formula (I) may further comprise a linker attached at the C-terminal to form an oligopeptide complex. Any kind of linker may be used as long as it does not prevent the oligopeptide complex from binding to dioxin. For example, amino acids, peptides, monosaccharides, disaccharides, polysaccharides, polyethers, supports, spacers, and the like may be mentioned. Useful supports and spacers include those exemplified above.

Alternatively, the oligopeptide shown in Formula (I) may be taken as a repeating unit, and two or more of the repeating units may be linked and used as a linearly-linked peptide. In forming the linearly-linked peptide, two or more oligopeptides as a repeating unit may be linked either via a spacer or directly. Any kind of spacer may be used as long as it does not prevent the linearly-linked peptide from binding to dioxin. For example, a polyethylene oxide chain may be mentioned. The linearly-linked peptide may also be attached to any of the supports exemplified above.

Dioxins detected or quantified by the dioxin binding peptide of the invention include those having toxic equivalency factors, such as polychlorinated dibenzoparadioxins (PCDDs), polychlorinated dibenzofurans (PCDFs) and coplanar PCBs (see FIG. 7). Examples of dioxins having toxic equivalency factors include PCDDs, such as 2,3,7,8-TeCDD, 1,2,3,7,8-PeCDD, 1,2,3,4,6,7,8-HpCDD, 1,2,3,4,6,7,8,9-OCDD, 1,2,3,4,7,8-HxCDD, 1,2,3,6,7,8-HxCDD, 1,2,3,7,8,9-HxCDD etc.; and PCDFs such as 2,3,7,8-TCDF, 2,3,4,7,8-PeCDF, etc. Examples of coplanar PCBs include 3,3′4,4′5-PeCB and the like. All such dioxins can be detected using the dioxin binding peptide of the invention. For detecting or quantifying a dioxin having a toxic equivalency factor other than those mentioned above, a calibration curve may be created based on the amount of any dioxin that can be detected or quantified by the dioxin binding peptide of the invention, so as to estimate the concentration of the dioxin.

The present method of detection or quantification is applicable to any kind of test sample for which dioxin is detected or quantified. Among the examples of test samples are air, soil, incinerated ash, water test samples from seawater and rivers as well as biological test samples such as blood, urine, saliva and maternal milk. A test sample may be subjected to dilution, extraction, elution, filtration and other like pretreatment, if necessary, before the application of the method of detection or quantification.

### Method of Detecting or Quantifying Dioxin

The method of detecting or quantifying dioxin using the dioxin binding peptide of the invention encompasses a method comprising the steps of:

(1) bringing a peptide immobilized support formed by linking the dioxin binding peptide of the invention into contact with a labeled dummy and a test sample which may contain dioxin; and

(2) detecting or quantifying the dioxin based on the amount of the labeled dummy bound to the support as determined in Step (1).

The term “quantifying dioxin” herein means measuring the concentration of dioxin contained in a test sample. The term “detecting dioxin” herein means determining whether dioxin is present or not based on the presence or absence of a signal from the labeled dummy

The labeled dummy for use in the invention denotes a dummy compound for dioxin which is labeled with a labeling material, and the compound is capable of binding to the dioxin binding peptide of the invention. This labeled dummy further denotes a dummy compound whose binding capability to the dioxin binding peptide of the invention is equal to or less than that of dioxins and is superior to that of compounds analogous to dioxins and dioxins not having toxic equivalency factors.

Compounds useful as such a labeled dummy compound are 3,4-dichlorophenol, 3,4-dibromophenol, 3,4,5-trichlorophenol, 2,3,4-trichlorophenol and the like. Derivatives of dioxins may also be used.

The dummy compound can be labeled by a variety of methods usually employed. For example, the dummy compound may be labeled with a fluorescent substance, radioisotope, enzyme or the like, or a dye or colorant such as a gold colloid or dyed latex. The labeling material is preferably a material which does not degrade the binding capability of the dummy compound to be labeled to the dioxin binding peptide of the invention.

Examples of fluorescent substances are NBD, FITC, NDA, OPA, RTIC, DTAF and the like. NBD or other fluorescent substance having an analogous structure is preferably used for 3,4-dichlorophenol.

Useful examples of radioisotopes are ³²P, ³H, ³⁵S, ¹²⁵I, ¹⁴C and the like.

Useful examples of enzymes include peroxidases, glucose oxidases, tyrosinases, acid phosphatases, alkaline phosphatases, and β-D-galactosidase and the like. When labeling with an enzyme, the compound to be labeled and the enzyme may be linked via a known spacer usually employed by those skilled in the art.

When using any of the above-mentioned enzymes, substrates which are colored by reaction with an enzyme are exemplified by chromogenic, fluorogenic and chemiluminescent substrates.

Examples of chromogenic substrates are 2,2′-azino-bis(3-ethylbenzthiazoline-6-sulphonic acid) (ABTS), 3,3′,5,5′-tetramethylbenzidine (TMB) or diaminobenzidine (DAB) in combination with hydrogen peroxide for peroxidases; 5-bromo-4-chloro-3-indolyl phosphate (BCIP) for alkaline phosphatases; and the like.

Examples of fluorogenic substrates are 4-methylumbelliferyl phosphate (4MUP) for alkaline phosphatases; 4-methylumbelliferyl-beta-D-galactoside (4MUG) for beta-D-galactosidase; and the like.

Examples of chemiluminescent substrates are 3-(2′-spiroadamantane)-4-methoxy-4(3″-phosphoryloxy)-phenyl-1,2-dioxetane disodium salt (AMPPD) for alkaline phosphatases; 3-(2′-spiroadamantane)-4-methoxy-4-(3″-β-D-galactopyranosyl)phenyll-1,2-dioxetane (AMGPD) for β-D-galactosidase; luminol or isoluminol in combination with hydrogen peroxide for peroxidases; and the like.

Such substrates can react with the labeled dummy before reaction with the test sample, or can be added at the time of reaction with the test sample in a solvent.

Useful coloring materials include metal colloid particles such as gold colloid particles; dyed latex particles obtained by coloring latex particles with a dye or pigment such as sudan blue, sudan red IV, sudan III, oil orange, quinizarin green and the like.

The method of detecting or quantifying dioxin according to the invention is described below using 3,4-dichlorophenol fluorescently labeled with NBD (NBD-labeled dichlorophenol) as a labeled dummy. This example, however, serves only to illustrate the invention, and persons skilled in the art may practice the method using other labeled dummy compounds by suitably changing or modifying the conditions, where necessary, based on the example of 3,4-dichlorophenol illustrated below.

In a vial or the like filled with a solvent, NBD-labeled dichlorophenol is bound to the dioxin binding peptide of the invention to enable identification by fluorescent emission. The dioxin binding peptide of the invention bound with the NBD-labeled dichlorophenol is then mixed with a test sample which may contain dioxin. Dioxin has a binding capability equal to that of 3,4-dichlorophenol. Therefore, dioxin binds competitively with 3,4-dichlorophenol to the dioxin binding peptide of the invention. The fluorescence intensity is decreased upon dissociation of the NBD-labeled dichlorophenol from the dioxin binding peptide, which indicates the presence of dioxin in the test sample (see FIG. 2).

In the present method of detection or quantification, the order of mixing the oligopeptide, labeled dummy and test sample which may contain dioxin is not particularly limited. Accordingly, the test sample which may contain dioxin and the dioxin binding peptide of the invention may be mixed before the labeled dummy is mixed thereto, or all of them may be mixed simultaneously.

In one example of the present method of detecting or quantifying dioxin, about three peptide immobilizing supports (dioxin-binding peptide beads of (3) in FIG. 1) were prepared per 1 μl of the test sample which may contain dioxin ((2) in FIG. 1) and 1 μl of a 1 μM labeled dummy (NBD-labeled dichlorophenol of (1) in FIG. 1) , and these were reacted in 1 ml of a 10 mM phosphate buffer solution (pH: 8) containing 20-30% 1,4-dioxane, after which the amount of the labeled dummy was detected or quantified (see FIG. 1).

It is also possible to employ a method in which the test sample is passed through a column packed with the peptide immobilizing supports (e.g., beads) such that the test sample is brought into contact with the supports. The invention, however, is not limited to such a method.

When the peptide immobilizing supports in bead form are used for the evaluation, the number of supports used is preferably about 1 to about 15, and more preferably about 1 to about 10, for example, per 1 ml of a test sample which may contain dioxin and 1 ml of a 1 μM labeled dummy, the supports being beads having a substitution rate of 0.1 to 10 mmol/g and a particle diameter of 10 to 150 μm. In addition, about 100 to about 300 μmol/g of the peptides is preferably synthesized onto the beads.

The combination of the kinds of dioxin binding peptide of the invention, labeling material, labeled compound and spacer is not particularly limited. For example, when the oligopeptides of DB2 shown in SEQ ID No. 3 are used for detecting or quantifying dioxin, it is preferable to use DB2 oligopeptides which are solid-phase synthesized on beads with a spacer of a polyethylene oxide chain interposed therebetween. In this case, NBD labeled 3,4-dicholorophenol can be used as a labeled dummy.

An organic solvent, such as 1,4-dioxane, can be used as a solvent, examples of which include 1,3-dioxane, dimethylformamide, N-methyl pyrolidone and the like. With 1,4-dioxane, for example, a phosphate buffer solution containing about 10 to about 50%, preferably about 20 to about 30%, of 1,4-dioxane can be used.

The method of detecting or quantifying the labeled dummy is not particularly limited. For example, for fluorescent labeling or dye staining, detection or quantification can be performed by recording microscopic images. When using other labeling materials, evaluation may be performed by a method suited to each material. For labeling with a fluorescent material, the amount of the labeled dummy can also be detected or quantified by measuring the amount of fluorescence emitted from the labeling material which is left unbound in the supernatant.

At this point, the dioxin can be quantified by establishing a calibration curve using a sample of known concentration. Moreover, the results of competitive quenching (FIGS. 6 (A) and (B)) described in the Examples of the present invention can be utilized as calibration curves for dioxin quantification using on-bead fluorescent competitive quenching. During quantification, the staining time necessary for clearly quantifying the competitive quenching is about 12 to about 30 hours, and more preferably about 15 to about 30 hours.

### Method of Extracting Dioxin

The oligopeptide of the invention that is capable of binding to dioxin is useful as a dioxin binding material in a simple pretreatment for determining and analyzing dioxin. The oligopeptide is also useful in extracting dioxin.

The extraction of dioxin according to the invention is performed by dissolving a test sample which may contain dioxin in a suitable solvent; adding a support having the oligopeptide of the invention to the resulting solvent; incubating the mixture for a predetermined period at room temperature to bind the dioxin to the support; and separating the dioxin from the support using a suitable solvent to recover the dioxin. It is thus possible to remove the dioxin from a test sample in a selective manner by the method of extracting the dioxin using the dioxin binding peptide of the invention. The dioxin extraction can be confirmed by measuring the amount of residues in the solution using a gas chromatography mass spectrometer.

Solvents useful for separating dioxin from the peptide immobilizing support include organic solvents such as 1,4-dioxane. For example, 1,3-dioxane, dimethyl formamide, N-methyl pyrolidone and the like can also be mentioned. The concentration of the solvent for use in separating dioxin from the peptide immobilizing support is about 50 to about 100%, preferably about 80 to about 100%, and more preferably about 100%.

In one example of the present method, 100 μl of a test sample solution which may contain dioxin and which is prepared using a 10 mM phosphate buffer solution (pH: 8) containing 30% 1,4-dioxane is put into a vial; and about one-hundred dioxin binding beads as the peptide immobilizing support of the invention are then added to the solution. The resulting solution is then incubated with mild shaking for 10 hr at room temperature, which causes the dioxin to bind to the beads.

When beads are used as the peptide immobilizing support for extracting dioxin, the number of beads used is preferably about 50 to about 500, and more preferably about 50 to about 300, for example, per 100 μl of the test sample solution which may contain dioxin which is prepared using a 10 mM phosphate buffer solution (pH: 8) containing 30% 1,4-dioxane, the beads having a substitution rate of 0.1 to 10 mmol/g and a particle diameter of 10 to 150 μm. In addition, about 100 to about 300 μmol/g of the dioxin binding peptides is preferably synthesized onto the beads.

The solution containing the beads and test sample is incubated for about 5 to about 20 hr, and preferably about 10 hr.

The dioxin binding peptide of the invention is also useful in pretreatment for quantifying and analyzing dioxin. Use of the dioxin binding peptide in pretreatment enables the dioxin to be separated from a test sample containing impurities, thereby preventing the impurities from affecting the subsequent determination and analysis. Moreover, the presence and absence of dioxin in the test sample can be easily confirmed using the dioxin binding peptide.

The dioxin binding peptide of the invention is highly selective to dioxin, and is therefore capable of detecting or quantifying dioxin in a test sample containing impurities. In addition, the dioxin binding peptide of the invention enables the selective extraction of dioxin from a test sample containing impurities, and is useful in simple pretreatment for quantifying and analyzing dioxin.

The dioxin binding peptide of the invention can be produced by peptide chemical synthesis at a lower cost than the conventional cost. Furthermore, the use of the dioxin binding peptide of the invention eliminates the need for pretreatment and the like, thus permitting the rapid and simple detection or quantification of dioxin.

### BRIEF DESCRIPTION OF THE DRAWINGS

FIG. 1 shows steps of detecting or quantifying dioxin.

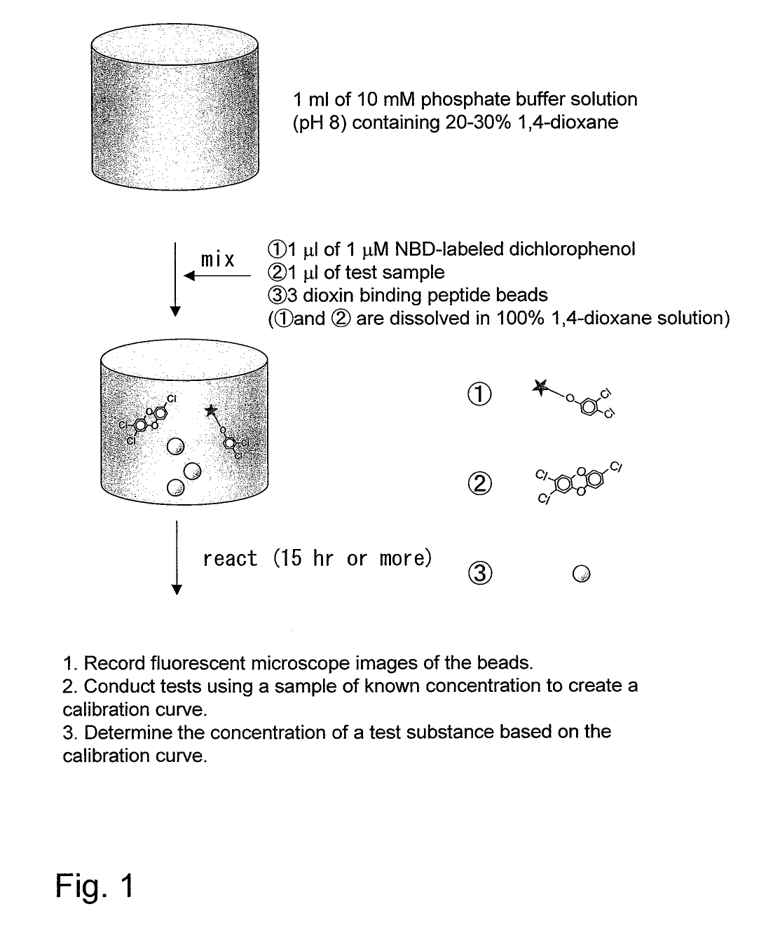

FIG. 2 shows steps of screening dioxin binding peptides.

FIG. 3 shows the structures of NBD-labeled dichlorophenol; 2,3,7-TriCDD; and 2,3,7,8-TeCDD.

FIG. 4 shows a fluorescence microscope image of a fluorescently stained dioxin binding bead, wherein the round object is the fluorescently stained dioxin binding bead.

FIG. 5 shows the results of on-bead competitive quenching tests for the DB2 peptide according to SEQ ID No. 3, wherein the round objects are the fluorescently stained dioxin binding beads.

FIG. 6 shows the results of on-bead competitive quenching tests for the DB2 peptide according to SEQ ID No. 3 conducted in a solvent containing 30% 1,4-dioxane, wherein graph (A) shows the fluorescence intensity of the beads, and graph (B) shows the quenching ratio of the beads, with the black circles representing the test results for 2,3,7,8-TeCDD, and the white circles representing the test results for 2,3,7-TriCDD.

FIG. 7 shows a graph of the relationship between dioxin concentration and the time it takes for fluorescent staining and competitive quenching, using 2,3,7-TriCDD having a concentration of 10 nM, wherein the black circles represent 10 nM NBD-labeled dichlorophenol; the black triangles represent 5 nM NBD-labeled dichlorophenol; the black squares represent 1 nM NBD-labeled dichlorophenol; the solid lines represent NBD-labeled dichlorophenol only; and the broken lines represent mixtures of NBD-labeled dichlorophenol at the aforementioned concentrations with 2,3,7-TriCDD.

FIG. 8 shows the structures of substituted amino acid side chains in the one amino acid-substituted peptide library.

FIG. 9 shows a graph illustrating the degree of NBD-labeled dichlorophenol staining of each peptide in the amino acid-substituted peptide library.

FIG. 10 shows the evaluation results of the dioxin binding capabilities of single residue-substituted peptides by the method of competitive dioxin binding on bead, wherein 1 Cha is the peptide according to SEQ ID No. 5; 5 Phg is the peptide according to SEQ ID No. 22; 5 Leu is the peptide according to SEQ ID No. 23; and 5 Nva is the peptide according to SEQ ID No. 24.

FIG. 11 shows the structures of test samples used in evaluating the peptide specificities by the method of competitive dioxin binding on bead.

FIG. 12 shows the results of binding specificity tests for the DB1 peptide according to SEQ ID No. 2 and the substituted peptides confirmed to have a capability of binding to 2,3,7,8-TeCDD equal to or more than that of the DB2 peptide according to SEQ ID No. 3 by the method of competitive dioxin binding on bead using a 30% 1,4-dioxane solvent, wherein 1 Cha is the peptide according to SEQ ID No. 5; 5 Phg is the peptide according to SEQ ID No. 22; 5 Leu is the peptide according to SEQ ID No. 23; and 5 Nva is the peptide according to SEQ ID No. 24; the horizontal axis of each graph representing the decrease in fluorescence intensity.

### BEST MODE FOR CARRYING OUT THE INVENTION

The present invention is further described in detail by the following Examples, which are not intended to limit the disclosure of the invention.

### EXAMPLE 1

### Obtaining Dioxin Binding Oligopeptides

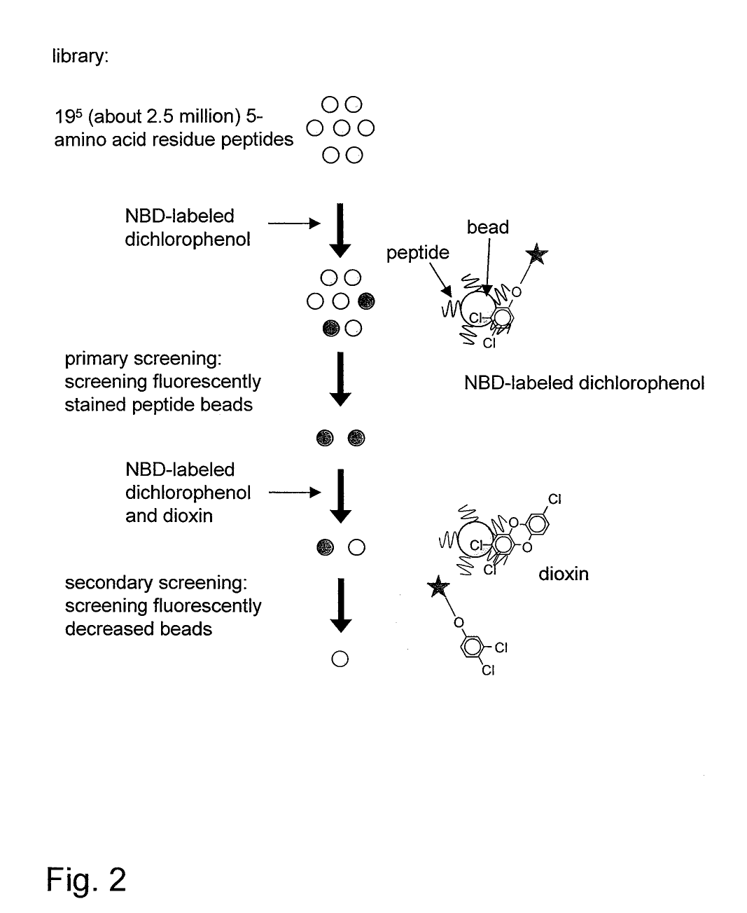

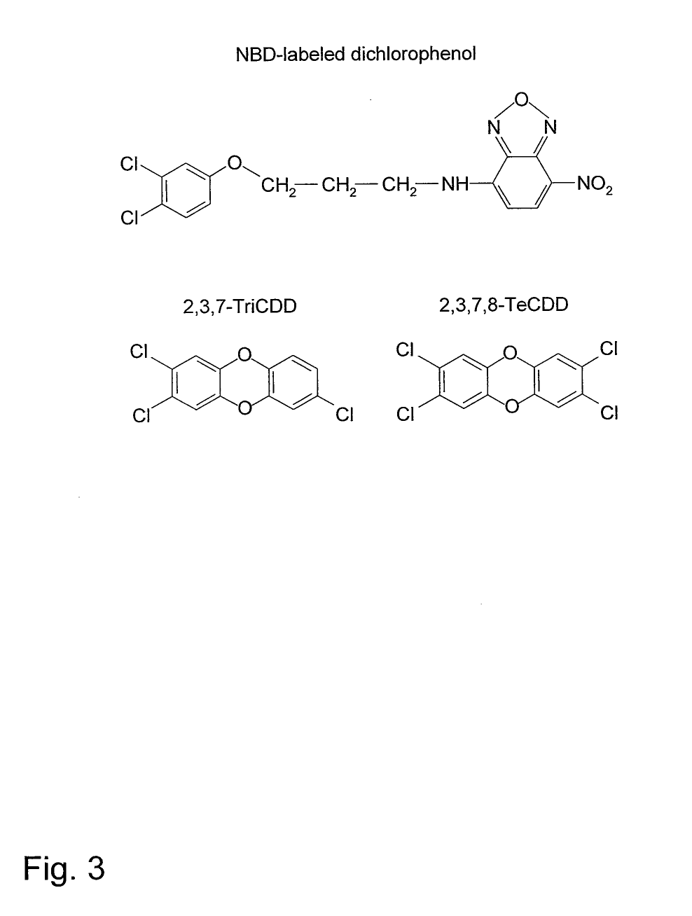

A peptide library was constructed by split-and-pool synthesis, one of the typical techniques of combinatorial chemistry, using beads for peptide solid-phase synthesis. According to the present method, a peptide of one kind of sequence is synthesized on a single bead. As shown in FIG. 2, screening consisted of two stages. Primary screening involved screening of fluorescently stained peptide beads, using a composite (FIG. 3) obtained by labeling 3,4-dichlorophenol, which has an analogous structure to dioxins, with NBD as a fluorescent material. Secondary screening involved screening of peptide beads fluorescently decreased by competition with 2,3,7-trichlorodibenzo-p-dioxin, i.e., peptide beads having an affinity to dioxin(s), from the peptide beads which were stained with the fluorescently labeled dichlorophenol.

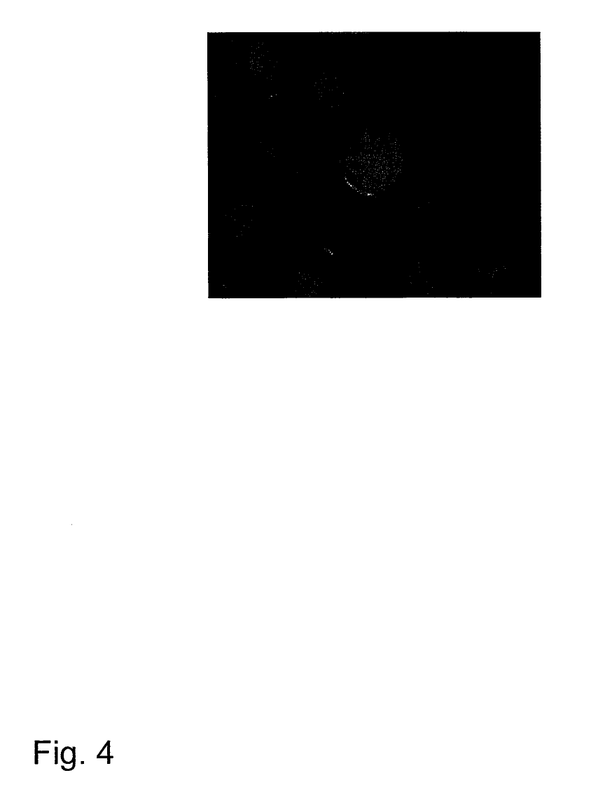

About 2.5 million peptide beads were used for screening, the number being equal to the number of combinations of all the sequences of peptides comprising 5 amino acid residues. The primary screening was conducted in a screening solvent (a 10 mM phosphate buffer containing 20% 1,4-dioxane (pH: 8)) containing 4 nM NBD-labeled dichlorophenol. 20 ml of the buffer solution was first mixed with about 50 mg of the peptide beads, and the mixture was subsequently incubated overnight, with mild shaking, in a petri dish at room temperature. A fraction of fluorescently stained peptide beads observed by a fluorescent microscope was collected with a micropipette. These peptide beads were transferred into a micro test tube containing 50 or 100% 1,4-dioxane, and were then incubated overnight at room temperature, i.e., washing with 50 or 100% dioxane overnight at room temperature. Peptide beads which were unable to be washed, on which the NBD-labeled dichlorophenol was unspecifically adsorbed, were excluded. Peptide beads that could be washed were re-stained with 1 nM NBD-labeled dichlorophenol. In order to measure the fluorescence intensity, fluorescent microscope images of the peptide beads were recorded with a digital camera (FIG. 4).

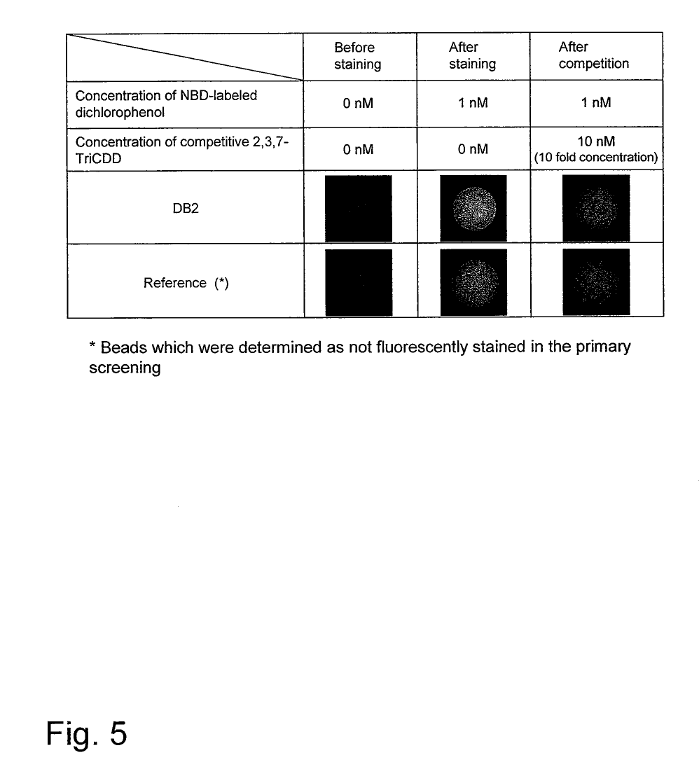

The aforementioned washable peptide beads were put into 1 ml of a screening solvent containing 1 nM NBD-labeled dichlorophenol and 10 or 100 nM 2,3,7-trichlorodibenzo-p-dioxin (2,3,7-TriCDD). The mixture was incubated overnight with mild shaking at room temperature, and was then transferred onto a glass petri dish, and fluorescent microscope images thereof were recorded. The obtained images were compared with the images recorded in the previous test so as to screen decreased beads. As shown in FIG. 5, two peptide beads were confirmed to be decreased under the competitive condition of NBD-labeled dichlorophenol (1 nM) with 10 fold concentration of 2,3,7-TriCDD (10 nM). The term “Reference” in FIG. 5 denotes the beads which were determined as not being fluorescently stained in the primary screening. The amino acid sequences of the peptides on the screened beads were determined with a protein sequencer. As a result, the amino acid sequences of the dioxin-binding peptide beads for which quenching by competition with 10 nM 2,3,7-TriCDD was confirmed proved to be Phe-Leu-Asp-Gln-Ile and Phe-Leu-Asp-Gln-Val. The Phe-Leu-Asp-Gln-Ile peptide bead was named DB1, and the Phe-Leu-Asp-Gln-Val peptide bead was named DB2.

### EXAMPLE 2

### Evaluation of Binding Capabilities of Dioxin Binding Peptides

Using dioxin-binding peptide beads, the dioxin binding capabilities of the peptides were evaluated in terms of affinity and specificity.

### Method of Competitive Dioxin Binding on Bead

1 ml of a screening solvent containing 4 nM NBD-labeled dichlorophenol and 0-100 nM of a substance to be detected was prepared in a glass vial, and then three dioxin-binding peptide beads were put into the screening solvent. The resulting mixture was incubated, with mild shaking, overnight at room temperature, and then fluorescent microscope images thereof were recorded. The average fluorescence intensity of each bead was calculated from the recorded images. Calculation of the average fluorescence intensity was performed with an image analysis/measurement software, “Image-Pro Plus” (Planetron, Inc.). When competitive binding of the dichlorophenol with a dioxin occurs, the fluorescence intensity of each bead decreases depending on the concentration of the dioxin. Such a method of measuring the dioxin concentration utilizing the quenching phenomenon of the beads was termed “the method of competitive dioxin binding on bead”.

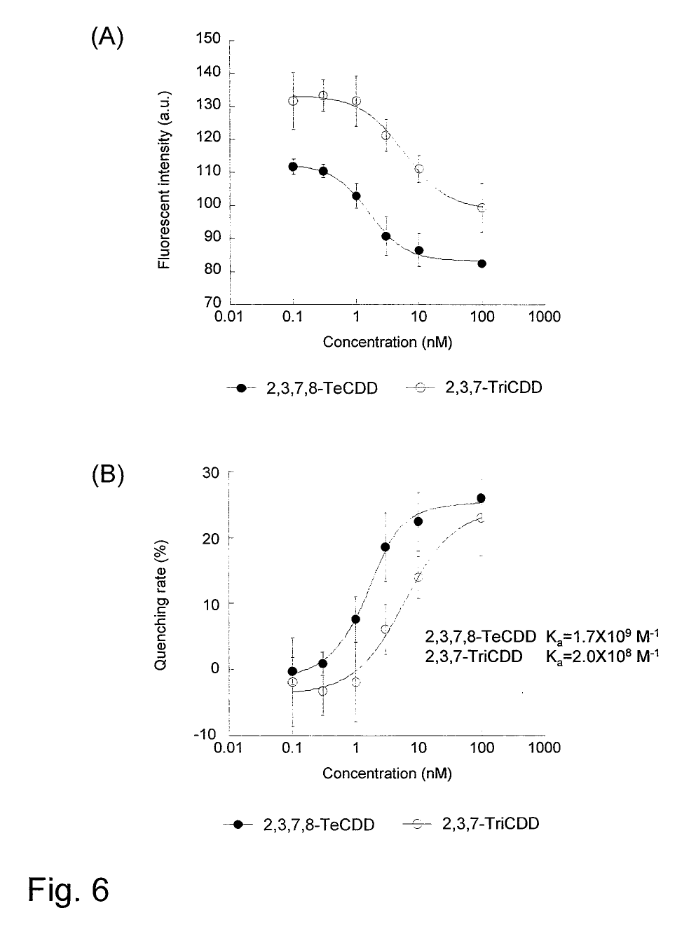

The DB2 peptide was evaluated for affinity to dioxins by the method of competitive dioxin binding on bead. FIGS. 6 (A) and 6 (B) show the results of tests conducted in a solvent containing 30% 1,4-dioxane. The relationship between dioxin concentration and average fluorescence intensity is plotted in FIG. 6 (A). As shown in the figure, declining sigmoid curves, which are characteristic of the competitive binding, were obtained. The curves were fitted using the four-parameter logistic equation, y=(a−d)/(1+(x/c)^{b})+d, which is an empirical equation typically employed in competitive ELISA (Eiji Ishikawa, “Enzyme Immunoassay (3rd Edition)”, IGAKU-SHOIN). Concentration-dependent quenching was observed for both 2,3,7-TriCDD and 2,3,7,8-TeCDD, and the results showed that 1 nM (about 0.3 ng/ml) 2,3,7,8-TeCDD can be detected using 30% 1,4-dioxane.

FIG. 6 (B) shows a graph which plots the quenching ratio of the beads determined from the average fluorescence intensity. The quenching ratio was calculated using the equation shown below. The beads were evaluated for affinity with the substances detected based on the values of quenching ratios.

quenching ratio=(fluorescence intensity 1−fluorescence intensity 2)/fluorescence intensity 1×100(%)

The binding constants of the DB2 peptide were calculated using the results of the above equation. Fitting was performed based on the theoretical equation for one-to-one binding of a receptor with a ligand as shown below:

Y=((Ymax/2e−9)*(1/2)*((2e−9+X*1e−9+1/Ka)−((2e−9+X*1e−9+1/Ka)\hat{]}2−4*2e−9*X*1e−9)̂0.5))−Ymin

Ka(binding constant)=10⁹(2,3,7,8-TeCDD), 10⁸(2,3,7-TriCDD)

Ymax (maximum quenching ratio)=0.25 (2,3,7,8-TeCDD), 0.25 (2,3,7-TriCDD)

Ymin (minimum quenching ratio)=−0.01 (2,3,7,8-TeCDD), −0.02 (2,3,7-TriCDD)

The results of fitting with these initial values showed that the peptides had affinities as high as 1.7×10⁹ M⁻¹ for 2,3,7,8-TeCDD and 2.0×10⁸ M⁻¹ for 2,3,7-TriCDD.

### EXAMPLE 3

### Method of Detecting Dioxin by Method of Competitive Dioxin Binding on Bead

A sample of known concentration (1 μl) was reacted with 1 μM NBD-labeled dichlorophenol (1 μl) and three dioxin-binding peptide beads in 1 ml of a 10 mM phosphate buffer solution (pH: 8) containing 20-30% 1,4-dioxane. After the reaction, fluorescence microscope images of the beads were recorded to establish a calibration curve.

Next, a test sample was reacted with the labeled dichlorophenol and dioxin binding beads, and then the obtained results were compared with the calibration curve, so as to give the dioxin concentration in the test sample.

The results of the competitive quenching shown in FIGS. 6 (A) and 6 (B) can be regarded as calibration curves for dioxin detection, the results being useful in detection using the method of competitive dioxin binding on bead.

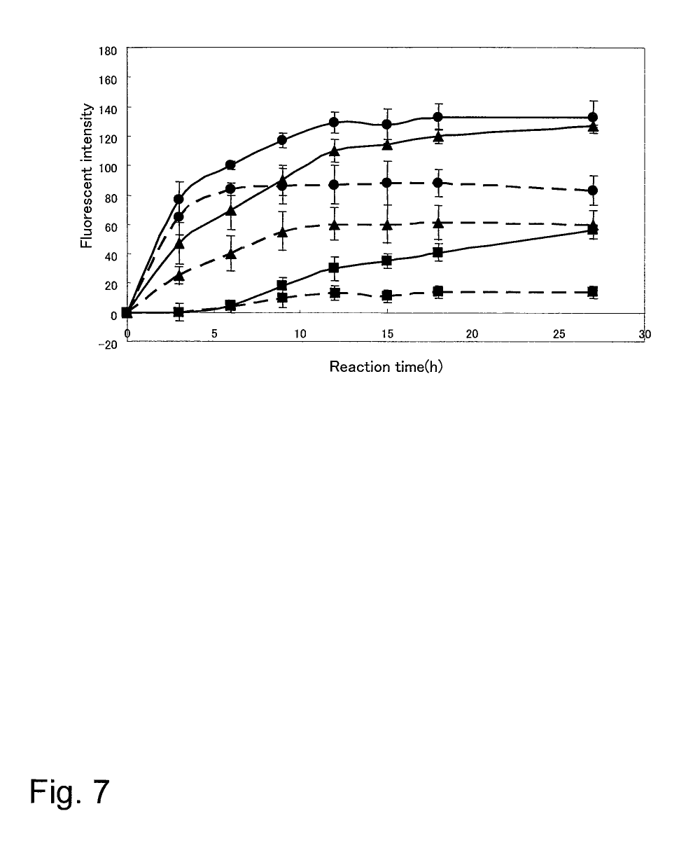

FIG. 7 shows the time required for staining. The results of the competition of 1-10 nM NBD-labeled dichlorophenol with 10 nM 2,3,7-TriCDD showed that incubation of 15 hr or more is necessary to detect clear quenching.

### EXAMPLE 4

### Analysis of Dioxin Binding Peptide Sequences

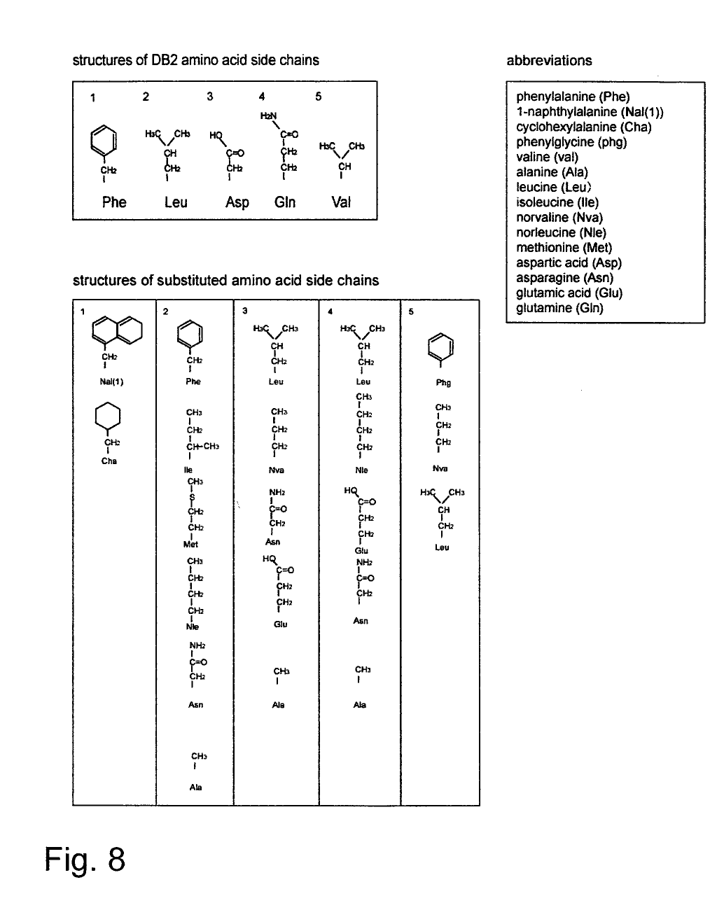

A library of twenty-one kinds of single amino acid-substituted peptides shown in FIG. 8 was constructed in order to evaluate the importance of each amino acid of the obtained oligopeptides, and to optimize the sequences. Those having analogous properties to the original amino acids, including unnatural amino acids, were used as substituting amino acids. DB1 and DB2 were added to these twenty-one kinds of peptides, and using these twenty-three kinds of peptides, evaluation was made based on staining with NBD-labeled dichlorophenol and quenching by dioxin binding. Table 1 shows all the sequences of the twenty-one amino acid-substituted peptides other than DB1 and DB2. The amino acid sequences in Table 1 are shown in the Sequence Listing with their sequence identification numbers corresponding to those shown in Table 1.

TABLE 1

SEQ ID No.

First Residue Substitution

| | | |
|---|---|---|
| SEQ ID No. 4 | Nal (1) | Leu Asp Gln Val |
| SEQ ID No. 5 | Cha | Leu Asp Gln Val |

Second Residue Substitution

| | |
|---|---|
| SEQ ID No. 6 | Phe Ala Asp Gln Val |
| SEQ ID No. 7 | Phe Phe Asp Gln Val |
| SEQ ID No. 8 | Phe Ile Asp Gln Val |
| SEQ ID No. 9 | Phe Met Asp Gln Val |
| SEQ ID No. 10 | Phe Nle Asp Gln Val |
| SEQ ID No. 11 | Phe Asn Asp Gln val |

Third Residue Substitution

| | |
|---|---|
| SEQ ID No. 12 | Phe Leu Ala Gln Val |
| SEQ ID No. 13 | Phe Leu Leu Gln Val |
| SEQ ID No. 14 | Phe Leu Nva Gln Val |
| SEQ ID No. 15 | Phe Leu Asn Gln Val |
| SEQ ID No. 16 | Phe Leu Glu Gln Val |

Fourth Residue Substitution

| | |
|---|---|
| SEQ ID No. 17 | Phe Leu Asp Ala Val |
| SEQ ID No. 18 | Phe Leu Asp Leu Val |
| SEQ ID No. 19 | Phe Leu Asp Nle Val |
| SEQ ID No. 20 | Phe Leu Asp Glu Val |
| SEQ ID No. 21 | Phe Leu Asp Asn Val |

Fifth Residue Substitution

| | |
|---|---|
| SEQ ID No. 22 | Phe Leu Asp Gln Phg |
| SEQ ID No. 23 | Phe Leu Asp Gln Leu |
| SEQ ID No. 24 | Phe Leu Asp Gln Nva |

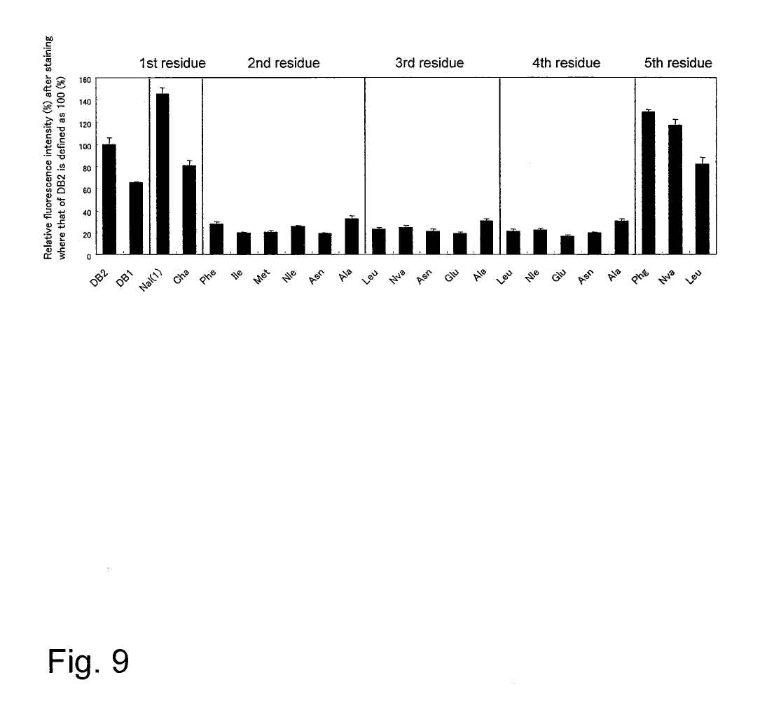

As shown in FIG. 9, because all the amino acid-substituted peptides tested, including those substituted with alanine, showed no fluorescent staining, the amino acids playing important roles in binding were found to be leucine as the second residue, aspartic acid as the third residue, and glutamine as the fourth residue. With 30% 1,4-dioxane, phenylalanine as the first residue could be replaced by 1-naphthylalanine or cyclohexylalanine. Valine or isoleucine as the fifth residue could be replaced by leucine or phenylglycine. With 30% 1,4-dioxane, the fifth residue could also be replaced by norvaline.

### EXAMPLE 5

### Sequence Suitable for Detecting Tetrachlorodibenzo-p-dioxin

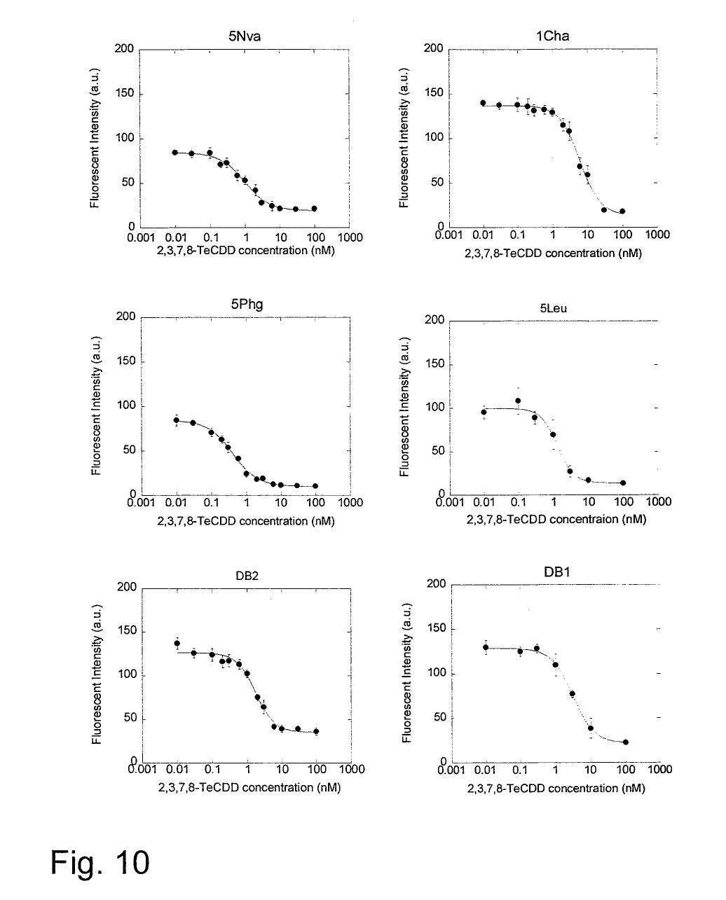

Tests were conducted on the above-mentioned peptides by the method of competitive dioxin binding on bead described in Example 2, using a 30% 1,4-dioxane solvent. The results confirmed that the peptide having cyclohexylalanine substituting for the first residue (N-terminal) amino acid (SEQ ID No. 5); peptide having phenylglycine substituting for the fifth residue (SEQ ID No. 22; 5 Phg); peptide having leucine substituting for the fifth residue (SEQ ID No. 23; 5 Leu); and peptide having norvaline substituting for the fifth residue (SEQ ID No. 24; 5 Nva) when stained each exhibit fluorescence intensity equal to or greater than that of the DB2 peptide (SEQ ID No. 3) (FIG. 10). All the four kinds of single residue-substituted peptides were evaluated for dioxin binding capability by the method of competitive dioxin binding on bead. The results showed that the peptide comprising 5 Phg (SEQ ID No. 22) is capable of detecting 0.15 nM (0.05 ng/ml) 2,3,7,8-TeCDD while providing a detection sensitivity about ten times greater than that of DB2.

### EXAMPLE 6

### Changes in Binding Specificities of Substituted Peptides

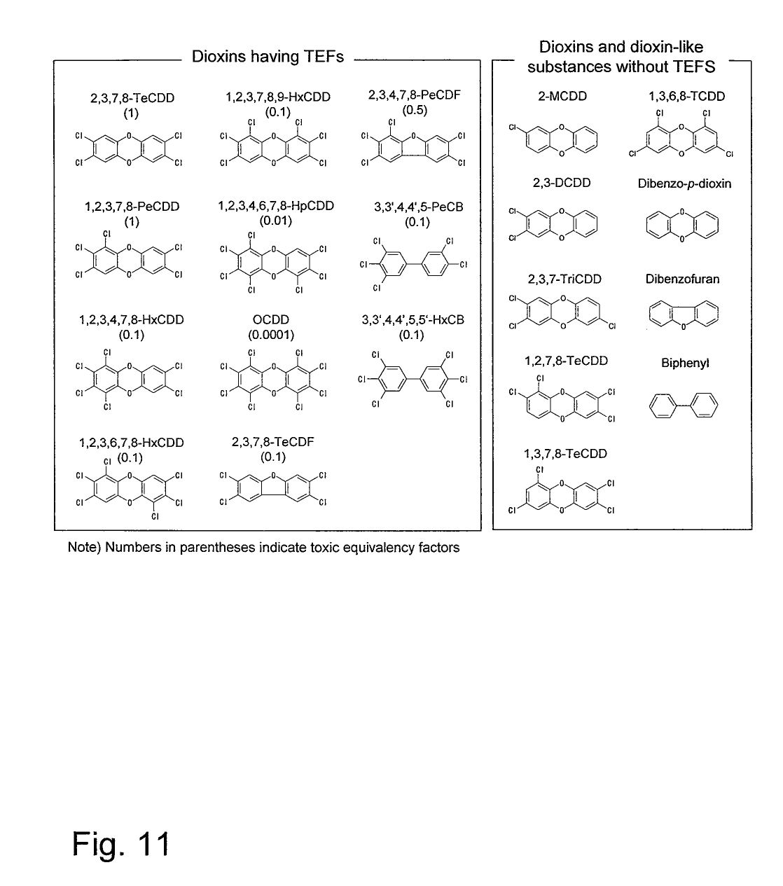

The specificity of the DB2 peptide was evaluated by the method of competitive dioxin binding on bead. FIG. 11 shows the structure of each of the substances to be detected used in evaluation.

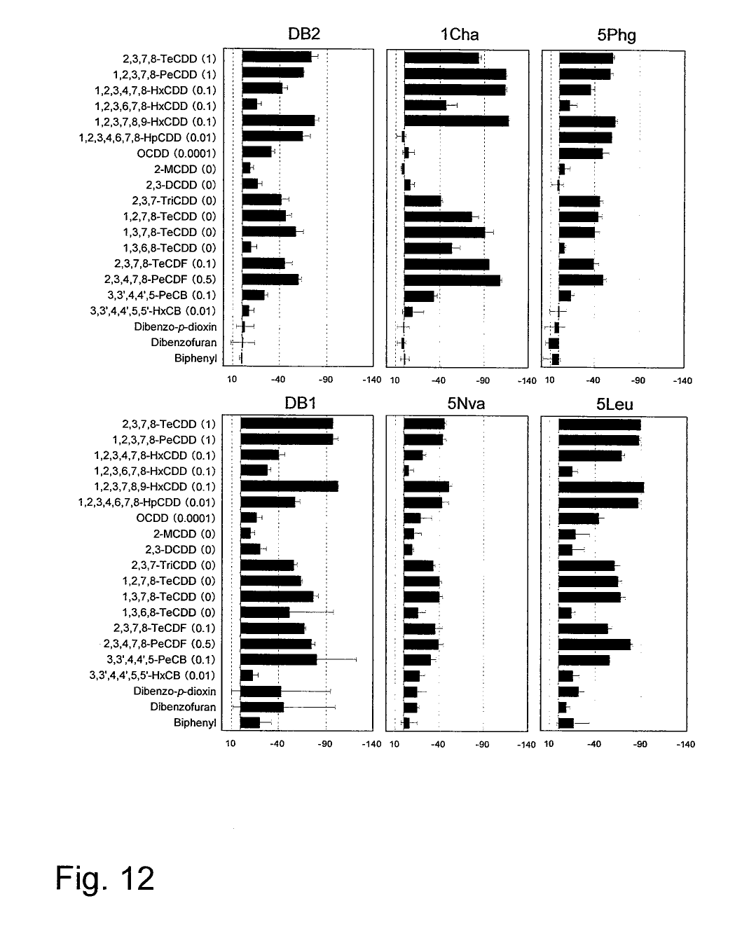

DB1 (SEQ ID No. 2) and the four substituted peptides (SEQ ID No. 5, 22, 23 and 24) confirmed to have a capability of detecting 2,3,7,8-TeCDD equal to or more than that of DB2 were evaluated for their binding specificities by the method of competitive dioxin binding on bead using a 30% dioxane solvent. Twenty substances in total, including dioxin isomers and other like substances, were used as the substances to be detected. For each peptide, “the change in fluorescence intensity” is shown (FIG. 12), which was obtained by subtracting the fluorescence intensity of the peptide after the addition of the substance to be detected from the maximum fluorescence intensity thereof when the substance had not been added. The greater the amount of change in the fluorescence intensity, the higher the affinity of the peptide to the substance. Consequently, the binding specificities of the substituted peptides to dioxin with a toxic equivalency factor (TEF) and the other tested chemicals varied compared to DB2.

1. An oligopeptide represented by Formula (I) shown below:
A₁-Leu-Asp-Gln-A₂-(X)ₙ (I)
where A₁ represents a hydrophobic amino acid residue having a side chain with a cyclic group; A₂ represents a hydrophobic amino acid residue having an aliphatic hydrocarbon group or an aromatic hydrocarbon group; n is zero or one; and X represents an amino acid residue.

2. A linearly-linked peptide formed by linking two or more oligopeptides represented by Formula (I) as a repeating unit via a spacer, if necessary.

3. An oligopeptide complex formed by using a linker to the C-terminal of the oligopeptide according to claim 1.

4. An oligopeptide according to claim 1, wherein A₁ is represented by Formula (II) shown below: 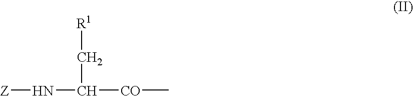
where R¹ represents a cyclic group; and Z represents a hydrogen atom, an alkyl group or an acyl group.

5. An oligopeptide according to claim 1, wherein A₁ is phenylalanine, 1-naphthylalanine, or cyclohexylalanine.

6. An oligopeptide according to claim 1, wherein A₂ is represented by Formula (III) shown below: 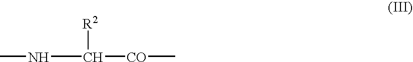
where R² is an alkyl or aryl group.

7. An oligopeptide according to claim 1, wherein A₂ is valine, norvaline, leucine, or phenylglycine.

8. An oligopeptide according to claim 1, comprising Phe-Leu-Asp-Gln-Ile.

9. An oligopeptide according to claim 1, comprising Phe-Leu-Asp-Gln-Val.

10. An oligopeptide according to claim 1, comprising Phe-Leu-Asp-Gln-Phg, where Phg represents a phenylglycine residue.

11. A process for detecting or quantifying dioxin in a sample wherein the oligopeptide, linearly-linked peptide and oligopeptide complex according to claim 1 is used for detecting or quantifying dioxin in said sample.

12. A peptide immobilizing support formed by linking
(a) the oligopeptide according to claim 1, (b) a linearly-linked peptide formed by linking two or more oligopeptides according to claim 1 or (c) an oligopeptide complex formed by using a linker to the C-terminal of the oligopeptide according to claim 1
to (d) a support.

13. A peptide immobilizing support according to claim 12, wherein the support is a bead.

14. A method of detecting or quantifying dioxin is selected from the group consisting of
(A) the method comprising the steps of:
(1) bringing the peptide immobilizing support according to claim 12 into contact with a labeled dummy and a test sample which may contain dioxin; and
(2) detecting or quantifying dioxin based on the amount of the labeled dummy bound to the support which is determined in Step (1); and
(B) the method comprising the steps of:
(1) bringing the peptide immobilizing support according to claim 12 into contact with a test sample containing dioxin to bind the dioxin to the support: and
(2) separating the dioxin bound to the support obtained in Step (1) using a solvent.

15. A method according to claim 14, wherein in (A) the labeled dummy is NBD-labeled 3,4-dichlorophenol.

16. A method of extracting dioxin according to claim 14, wherein said method is (A).

17. A method of extracting dioxin according to claim 14, wherein said method is (B).


SEQUENCE LISTING (24 sequences)

SEQ ID NO: 1 - protein, 6 aa, Artificial Sequence
  Description of Artificial Sequence Synthetic peptide
XLDQXX

SEQ ID NO: 2 - protein, 5 aa, Artificial Sequence
  Description of Artificial Sequence Synthetic peptide
FLDQI

SEQ ID NO: 3 - protein, 5 aa, Artificial Sequence
  Description of Artificial Sequence Synthetic peptide
FLDQV

SEQ ID NO: 4 - protein, 5 aa, Artificial Sequence
  Description of Artificial Sequence Synthetic peptide
XLDQV

SEQ ID NO: 5 - protein, 5 aa, Artificial Sequence
  Description of Artificial Sequence Synthetic peptide
XLDQV

SEQ ID NO: 6 - protein, 5 aa, Artificial Sequence
  Description of Artificial Sequence Synthetic peptide
FADQV

SEQ ID NO: 7 - protein, 5 aa, Artificial Sequence
  Description of Artificial Sequence Synthetic peptide
FFDQV

SEQ ID NO: 8 - protein, 5 aa, Artificial Sequence
  Description of Artificial Sequence Synthetic peptide
FIDQV

SEQ ID NO: 9 - protein, 5 aa, Artificial Sequence
  Description of Artificial Sequence Synthetic peptide
FMDQV

SEQ ID NO: 10 - protein, 5 aa, Artificial Sequence
  Description of Artificial Sequence Synthetic peptide
FXDQV

SEQ ID NO: 11 - protein, 5 aa, Artificial Sequence
  Description of Artificial Sequence Synthetic peptide
FNDQV

SEQ ID NO: 12 - protein, 5 aa, Artificial Sequence
  Description of Artificial Sequence Synthetic peptide
FLAQV

SEQ ID NO: 13 - protein, 5 aa, Artificial Sequence
  Description of Artificial Sequence Synthetic peptide
FLLQV

SEQ ID NO: 14 - protein, 5 aa, Artificial Sequence
  Description of Artificial Sequence Synthetic peptide
FLXQV

SEQ ID NO: 15 - protein, 5 aa, Artificial Sequence
  Description of Artificial Sequence Synthetic peptide
FLNQV

SEQ ID NO: 16 - protein, 5 aa, Artificial Sequence
  Description of Artificial Sequence Synthetic peptide
FLEQV

SEQ ID NO: 17 - protein, 5 aa, Artificial Sequence
  Description of Artificial Sequence Synthetic peptide
FLDAV

SEQ ID NO: 18 - protein, 5 aa, Artificial Sequence
  Description of Artificial Sequence Synthetic peptide
FLDLV

SEQ ID NO: 19 - protein, 5 aa, Artificial Sequence
  Description of Artificial Sequence Synthetic peptide
FLDXV

SEQ ID NO: 20 - protein, 5 aa, Artificial Sequence
  Description of Artificial Sequence Synthetic peptide
FLDEV

SEQ ID NO: 21 - protein, 5 aa, Artificial Sequence
  Description of Artificial Sequence Synthetic peptide
FLDNV

SEQ ID NO: 22 - protein, 5 aa, Artificial Sequence
  Description of Artificial Sequence Synthetic peptide
FLDQX

SEQ ID NO: 23 - protein, 5 aa, Artificial Sequence
  Description of Artificial Sequence Synthetic peptide
FLDQL

SEQ ID NO: 24 - protein, 5 aa, Artificial Sequence
  Description of Artificial Sequence Synthetic peptide
FLDQX